(19) United States
(12) Patent Application Publication
(10) Pub. No.: US 2007/0167992 A1
(43) Pub. Date: Jul. 19, 2007

(54) Method and apparatus for reducing preterm labor using neuromodulation

(75) Inventor: Michael Edward Carley, Dallas, TX (US)

Correspondence Address: CHALKER FLORES, LLP, 2711 LBJ FRWY, Suite 1036, DALLAS, TX, 75234, US

(73) Assignee: Baylor Research Institute, Dallas, TX (US)

(21) Appl. No.: 11/335,395

(22) Filed: Jan. 18, 2006

(51) Int. Cl. A61N 1/02

(52) U.S. Cl. 607/46

(57) ABSTRACT

The present invention includes an apparatus, kit and method for providing neural stimulation to reduce preterm labor contractions and thereby reduce subsequent preterm births. The present invention includes one or more implantable electrodes adapted for electrical communication with one or more sacral nerve roots and an electrical energy generator to produce one or more electrical signals in electrical communication with the one or more implantable electrodes.

### TECHNICAL FIELD OF THE INVENTION

The present invention relates in general to the field of electronically stimulating the efferent and/or afferent nerves, and more particularly, to the electro-stimulation of the sacral nerves to reduce preterm labor and preterm delivery during pregnancy.

### BACKGROUND OF THE INVENTION

Without limiting the scope of the invention, its background is described in connection with the alteration of the sensory input and output of the nervous system using electro-stimulation of the spinal nerve roots (e.g., the sacral nerve roots) and other nerve bundles for reductions in preterm labor contractions and preterm delivery during pregnancy, as an example.

Preterm labor and preterm delivery during pregnancy represents one of the greatest causes of morbidity for infants in the United States. Generally, preterm labor is defined as labor that occurs before completion of the 37^{th} week of gestation and the fetus is unable to live outside the womb. Preterm delivery affects approximately one in every eight to ten births and is the cause of at least 75 percent of the neonatal deaths. Additionally, about 20% of the premature infants that survive preterm delivery die in the first month.

In addition, premature infants that survive face a number of serious health concerns, e.g., low birth weight, breathing problems and underdeveloped organs and organ systems. As a result, infants born prematurely need to stay in the hospital for extended periods of time and require specialized equipment to allow their health to stabilize. In addition to complications at birth, infants who survive have an increased risk for certain life-long health affects, e.g., cerebral palsy, blindness, lung diseases, learning disabilities and developmental disabilities. Some research also suggests that babies born prematurely are at higher risk for certain health problems as they age.

In addition to the extreme physical and emotional strain preterm labor and delivery has on the family it also imparts a significant financial burden to the family and society. The hospitalization costs for preterm infants (e.g. antepartum maternal care, the neonatal intensive care and the immediate care of the prematurely born infant) can easily exceed $500,000 per case. In addition, the costs for specialized care of the premature newborn continue to accrue after discharge from the hospital. In some instances, specialized care must be provided for the remained of the child's life, e.g., life long handicaps.

Although, preterm labor and preterm delivery often results in death, few medical advances have been made in the medical community to reduce the number of preterm deliveries. Current approaches to the prevention of preterm birth rely in part on identifying a group of women to whom special attention can be directed. The healthcare providers provide education regarding the signs and symptoms associated with preterm labor and provide monitoring to identify preterm birth conditions and preterm labor. In an effort to stop preterm labor, the health care providers take steps to stop labor if it starts before 37^{th} weeks of pregnancy. One possible reason for the limited treatment options is the limited information and poor understanding regarding the pathophysiology of preterm labor and preterm delivery. The causes of preterm labor and preterm delivery are thought to be multifactoral. Common methods for trying to stop labor include behavioral modifications such as bed rest and medications that relax the muscles in the uterus involved with labor and delivery.

For example, the prevention of preterm births is taught in U.S. Pat. No. 6,375,970 issued to Bieniarz, which teaches materials and methods for reducing the incidence of preterm birth involving the use of polymeric compositions. A uterine cervix and intrauterine polymeric system on or adjacent to the chorioamniotic membrane with a polymeric material. The chorioamniotic membrane may have an elongation at rupture similar to or greater than that of chorioamniotic membrane or may be characterized by an elongation at rupture. The polymeric material may be adherent to the chorioarniotic membrane having an elastic modulus, a tensile stress and a tensile modulus that provides sufficient physical support to reduce stretching of the chorioamniotic membrane into the uterine cervix during pregnancy. The force required to rupture said polymeric material is similar to or greater than that required to rupture chorioamniotic membrane. The polymeric material may also form a physical barrier preventing migration of vaginal microbes into the uterus.

Another example of a method for the treatment of preterm labor is taught in U.S. Pat. No. 5,929,071 issued to Salata, Jr., which teaches administering a pharmacologically effective amount of a selective modulator of IKs. Further, a method of stopping labor prior to vaginal or cesarean delivery and treatment of dysmenorrhea is taught and includes administration of a pharmacologically effective amount of a modulator of IKs.

The foregoing problems have been recognized for many years and while numerous solutions have been proposed, none of them adequately address all of the problems in a single device or method.

### SUMMARY OF THE INVENTION

The present inventor recognized a need for specific, reliable, effective method for treating preterm labor and preterm delivery through the neuromodulation of the nerves associated with the spinal cord to reduce preterm labor contractions during pregnancy. The device discloses herein extend the pregnancy term through reducing preterm contractions and preterm delivery which in turn reduces infant morbidity and the cost associated with preterm delivery care and hospital stays.

More particularly, a method, apparatus and kit are provided that induce neural stimulation to reduce preterm labor contractions. The present invention includes one or more implantable electrodes adapted for electrical communication with one or more sacral nerve roots and an electrical energy generator to generate one or more electrical pulses in electrical communication with the one or more implantable electrodes.

In addition, the present invention includes a neural stimulation kit for the reduction of preterm labor contractions including one or more percutaneous electrodes adapted for electrical communication with one or more nerve roots and an electrical energy generator to produce one or more electrical pulses in electrical communication with the one or more implantable electrodes.

The present invention also provides a neuromodulation device for the reduction of preterm labor contractions having one or more percutaneous electrodes adapted for electrical communication with one or more dura layers surrounding one or more sacral nerve roots and an electrical energy generator to generate one or more electrical pulses in electrical communication with the one or more implantable electrodes.

For example, the present invention includes an implantable neurostimulation apparatus to reduce preterm labor contractions. The apparatus includes one or more implantable electrodes adapted for electrical communication with one or more sacral nerve roots and an electrical energy generator for generating one or more electrical signals in electrical communication with the one or more implantable electrodes.

The present invention includes a method of neuron stimulation to reduce preterm labor by connecting one or more electrodes, under the control of a neuron stimulation apparatus including an electrical energy generator, to one or more sacral nerves. The one or more electrodes are stimulated through the conduction of the one or more electrical pulses to the one or more electrodes.

The present invention also provides a method, apparatus and kit that induce neural stimulation to modulate contractions and/or pain. The present invention includes one or more implantable electrodes adapted for electrical communication with one or more sacral nerve roots and an electrical energy generator to generate one or more electrical pulses in electrical communication with the one or more implantable electrodes.

### BRIEF DESCRIPTION OF THE DRAWINGS

For a more complete understanding of the features and advantages of the present invention, reference is now made to the detailed description of the invention along with the accompanying figure and in which:

FIG. 1 is a schematic view of the system connected to the electrodes which have been inserted into the body of the patient.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The terminology used and specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention provides a treatment for preterm labor and subsequent preterm delivery through the stimulation of the nerves of the spinal cord using neural stimulation electrodes and leads implanted in a patient. The implantation may be in the epidural space of the spinal canal or other nervous system structures to centrally and/or peripherally stimulate selected locations of the nerves of the spinal canal or other nervous system structures.

Generally, the central nervous system is protected by the thirty-three vertebrae of the spine. The vertebrae are sequentially divided into four regions that include the uppermost seven vertebrae referred to as the cervical vertebrae (C1-C7), the twelve thoracic vertebrae (T1-T12), the five lumbar vertebrae (L1-L5) and the five sacral vertebrae (S1-S5) respectively. The final four vertebrae are often fused together and referred to as the coccygeal vertebrae. The vertebrae of each of the four regions have similar general structures with slight structural differences.

The outside surface of the vertebrae is made of a relatively strong cortical bone layer, while the center is made of a weak cancellous bone. The vertebrae have basic structure that includes an anterior portion that is roughly cylindrical called the vertebral body with a superior surface that is concave transversely and convex antero-posterioly with prominent elevations on each side.

A triangular aperture (e.g., vertebral foramen) is formed in the vertebra to accommodate the spinal cord, meninges and associated vessels. The vertebral foramen is surrounded by the vertebral body and the posterior arch which includes the pedicles, the articular processes, the laminae and the spinous processes. The spinous processes project backwards from the junction of the laminae. Transverse processes arise anteriorly from the vertebral body and posteriorly from the articular processes to form the vertebral foramen.

The successive positioning of the vertebral bodies and the separation with intervertebral discs allows the vertebral foramen to surround the spinal cord. To allow the nerve roots of the spinal cord to connect to the peripheral nervous system, passageways (e.g., the neuroforamen) are formed on either side between an upper and lower vertebra and the intervertebral disc creating the height of the passageway. At a position below the Thoracic (T12) and first Lumbar (L1) vertebra the spinal cord ends at a structure called the Conus Medullaris. From the Conus Medullaris to the coccyx the spinal nerves form the Cauda Equina. Generally, there are thirty one pairs of spinal nerve roots that extend from the spinal cord and exit the neuroforamen either anteriorly (motor) or posteriorly (sensory). The spinal nerve roots are then connected to nerves that control the body's functions (e.g., the vital organs, sensation and movement) and transmit stimuli received from various sensory inputs (e.g., peripheral nerves) and initiate an appropriate response as a result of those internal and external stimuli.

The present inventor recognized that preterm labor and subsequent preterm delivery is in part influenced by neurological input to and from the uterus. The present inventor recognized the uterus is innervated principally by the involuntary or autonomic nervous system and that the sympathetic fibers arise from the thoracic and lumbar spinal segments (T10 to L2) and the parasympathetic fibers are derived from the sacral spinal segments (S2-S4). In addition, the inventor recognized that an implantable percutaneously inserted electrode (i.e., without requiring major surgery) may be used for reducing preterm labor contractions and subsequent preterm delivery. More specifically, the electrode is adapted for sacral spinal segments S2, S3 and/or S4 stimulation to reduce preterm contractions and in turn reduce preterm labor and subsequent delivery. The electrode has portions that are specifically provided for coupling the electrode to the adjacent spinal tissue and reduce the displacement of the electrode by normal bodily motion. The success of electrode placement and subsequent electronic stimulation is gauged by a decrease in the frequency and or intensity of uterine contractions with the goal of halting or slowing cervical change. The electrode remains in place until the risks of preterm delivery are no longer anticipated to represent significant fetal risk.

Generally, electrical energy has been applied to the nerves in the art (e.g., epidermis, spinal nerve roots, spinal cord and other nerve bundles) for many years in an effort to control chronic pain control; however, the interaction of the electrical energy and the tissue of the nervous system is not fully understood and therefore has limited its use in many areas. Many of the devices in the art use neuromodulation systems to mask pain and none related to controlling labor contractions.

Generally, the electrodes may be a percutaneous electrode, a laminotomy electrode or other electrode known to the skilled artisan. The percutaneous electrode requires a less-invasive implantation method and allows the positioning of multiple electrodes into the tissue to create an array of electrodes as needed, but the electrodes are prone to migration. In contrast, the laminotomy electrode requires major surgery and is to some extent preconfigured, but is less prone to migration during use.

The present inventor recognized that preterm delivery is in part influenced by neurological input to and from the uterus and the brain and an electrical field could be applied not only to mask pain, but to control the rate of labor contractions. More specifically, the present inventor recognized that the stimulation of the sacral nerve can result in the effective reduction of contractions associated with preterm labor and subsequent preterm delivery. The common method for introduction and nerve stimulation (e.g., using a percutaneous catheter or a laminotomy lead) is through the placement of electrodes external to the dura layer surrounding the spinal cord. The present invention includes the placement of one or more electrodes capable of delivering electrical energy in a position external to the dura layer surrounding the spinal cord in the S2, S3 and/or S4 region of the spine.

The present invention includes an implantable neurostimulation apparatus to reduce preterm labor having one or more implantable electrodes adapted for electrical communication with one or more sacral nerve roots and an electrical energy generator to produce one or more electrical pulses in electrical communication with the one or more implantable electrodes.

The present invention also includes an implantable neurostimulation apparatus to reduce pain and contractions associated with preterm labor. The apparatus includes one or more implantable electrodes adapted for electrical communication with one or more sacral nerve roots and an electrical energy generator to generate one or more electrical pulses in electrical communication with the one or more implantable electrodes.

The one or more implantable electrodes may be individually a wire, a rod, a filament, a ribbon, a cord, a tube, a formed wire, a flat strip or combinations thereof. The one or more implantable electrodes may be one or more percutaneous electrodes, one or more laminotomy electrodes or a combination thereof. For patient use, the device of the present invention will commonly use a pair to provide stimulation of the sacral nerves and nerve roots. The one or more implantable electrodes may be controlled individually or in series, parallel or any other manner desired. The one or more implantable electrodes may be held in position using any method known to the skilled artisan, including but not limited to stitches, epoxy, tape, glue, sutures or a combination thereof.

The one or more implantable electrodes are adapted for electrical communication with one or more sacral nerve roots; however, one or more implantable electrodes may also be positioned in the thoracic nerve roots and/or one or more lumbar nerve roots and in combination with the sacral nerve roots. When positioned in the sacral nerve roots the electrodes are positioned into the S2 sacral nerve roots, the S3 sacral nerve roots, the S4 sacral nerve roots and combinations thereof.

In addition, the present invention may be adapted for electrical communication with other nerves, e.g., dorsal scapular nerve; long thoracic nerve; lateral pectoral nerve; medial antebrachial cutaneous; thoracodorsal nerve; radial nerve; axillary nerve; subclavius nerve; suprascapular nerve; musculocutaneous nerve; median nerve; ulnar nerve; superficial peroneal nerve; deep peroneal nerve; lateral sural cutaneous nerve; spinal accessory nerve; saphenous nerve; lateral femoral cutaneous; obturator nerve; femoral nerve; common and proper digital nerves; anterior interosseus nerve; lateral antebrachial cutaneous; deep (motor) branch of the radial; posterior interosseus nerve; superficial (cutaneous) branch of the radial; posterior femoral cutaneous; superior gluteal nerve; piriformis nerve; sciatic nerve; inferior gluteal nerve; common peroneal nerve; tibial nerve; medial and lateral planter nerves; medial sural cutaneous; sural nerve; medial and lateral plantar nerves; deep (motor) branch of the ulnar; superficial (cutaneous) branch of the ulnar; and combinations thereof.

In addition, the present invention may be used to treat other stages of pregnancy, e.g., contraction pain, cesarean section and “post-term” pregnancies. For example, the present invention may be used to treat or reduce pain associated with uterine contractions or cesarean section through the stimulation of the nerves of the spinal cord to block pain signals using neural stimulation electrodes and leads implanted in a patient. The implantation may be in the epidural space of the spinal canal or other nervous system structures to centrally and/or peripherally stimulate selected locations of the nerves of the spinal canal or other nervous system structures.

In addition to preventing preterm labor contractions, the present invention may be used to stimulating labor in “post-term” pregnancies. For example, one or more electrodes adapted for electrical communication with one or more dura layers surrounding one or more sacral nerve roots and an electrical energy generator to generate one or more electrical pulses in electrical communication with the one or more electrodes. The electrical pulses of the electrodes result in the stimulation of labor contractions. In addition, the present invention may use the electrical pulses of the electrodes to modulate or reduce the pain and/or discomfort associated with labor and uterine contractions.

Another embodiment of the present invention may be used to stimulate or inhibit nerves in communication with other organs to modulate organ function or improve pain. For example, one embodiment of the present invention may be used to modulate bladder contractions using the electrical pulses of the electrodes to modulate the nerves involved in bladder contractions. Therefore, the present invention may be used to stimulate the contraction of the bladder or inhibit the contraction of the bladder.

In addition to humans, the present invention may be used to modulate the contraction and pain associated with various muscles and organs in other vertebrates and more specifically mammals, e.g., aardvarks; antelopes; armadillos; badgers; bats; bears; bobcats; buffalo; camels; cats; cheetahs; civet family; cougars; cows; coyotes; deer; dogs; dolphins; donkeys; elephant shrews; elephants; elk; ermine; ferrets; foxes; giraffes; goats; guanacos; hedgehogs; hippopotamuses; horses; hyenas; jaguars; leopards; lions; llamas; lynxes; manatees; marine mammals; marsupials; mink; moles; mongoose family; monotremes; moose; mules; mustelids; ocelots; pigs; pine marten; pinnipeds; primates; rabbits; raccoons; pandas; reindeer; caribou; rhinoceroses; rodents; sheep; skunks; sloths; solenodons; tapirs; tayras; tigers; vicunas; weasels; whales; wolverine; wolves; yaks; and zebras. For example the present invention may be use electro-stimulation of the sacral nerves to reduce preterm labor and preterm delivery during pregnancy in endangered or rare mammal species (e.g., panda, horses, etc.) having difficulties carrying to term.

The electrical energy generator controls the pulse waveform, the signal pulse width, the signal pulse frequency, the signal pulse phase, the signal pulse polarity, the signal pulse amplitude, the signal pulse intensity, the signal pulse duration and combinations thereof of the one or more electrical pulses. The electrical energy generator may be used to convey a variety of currents and voltages to the one or more implantable electrodes to affect the nerves. The electrical energy generator may be used to control numerous electrodes indeypendently or in various combinations as needed to provide stimulation. The skilled artisan will know the applicable ranges.

The signal may be constant, varying and/or modulated with respect to the current, voltage, pulse width, cycle, frequency, amplitude and so forth. For example, the current may range from generally from about 0.001 to about 1000 microampere (mA) and more specifically from about 0.1 to about 100 microampere (mA). Similarly, the voltage may range from about 0.1 millivolt to about 25 volts and about 0.5 to about 4000 Hz, with a pulse width of about 10 to about 1000 microseconds (mS). Furthermore, the type of stimulation may vary and involve different waveforms known to the skilled artisan. For example, the stimulation may be based on the H waveform found in nerve signals (i.e., Hoffinan Reflex) or different forms of interferential stimulation may be used.

The present invention may be used in conjunction with other electrodes (transcutaneous, percutaneous and peripherally implanted electrodes) and signal generators and in a variety of combinations. The present invention may also be used for transcutaneous neuromodulation of internal organs, muscles or surfaces. Transcutaneous neuromodulation includes the positioning of a surface electrode transcutaneously or partially transcutaneous. For example, the electrode may be placed in contact with the uterine muscle directly to modulate the stimulation and contractions. Generally, the signal may be constant, varying and/or modulated with respect to the current, voltage, pulse width, cycle, frequency, amplitude and so forth, e.g., the current may be between about 1 to 100 microampere (mA), about 10 V (average), about 1 to about 1000 Hz, with a pulse width of about 250 to about 500 microseconds (mS). Another example is the percutaneous neuromodulation using a needle-like electrode. Generally, the electrode is positioned in the soft tissues or muscles. Again, the signal may be constant, varying and/or modulated with respect to the current, voltage, pulse width, cycle, frequency, amplitude and so forth, e.g., the signal may have a 5-Hz frequency and a pulse width of 0.5 mS.

In addition, the electrical energy generator may include or be in communication with a CPU, a keyboard, a mouse, a touchpad, a touch screen, a Bluetooth wireless adaptor, an IR adaptor, a wi-fi adaptor, a RF adaptor, a blood pressure sensor, a heart rate sensor, an electrical activity sensor, a contraction sensor, a timer, speakers, a beeper, an input port, an output port, an IR sensor, a RF sensor, a biofeedback sensor, a LAN adaptor, wireless network adaptor and combinations thereof.

The present invention includes a neural stimulation kit for reduction of preterm labor including one or more percutaneous electrodes adapted for electrical communication with one or more nerve roots and an electrical energy generator to generate one or more electrical pulses in electrical communication with the one or more implantable electrodes. The one or more percutaneous electrodes may be provided individually or in pairs or sets such that the surgeon may select the best combination. The kit may also include a Touhy-like needle for insertion of the electrodes. Generally, the devices may be provided individually wrapped and/or pre-sterilized. The kit may also include an electrical energy generator that generates and/or controls the pulse waveform, the signal pulse width, the signal pulse frequency, the signal pulse phase, the signal pulse polarity, the signal pulse amplitude, the signal pulse intensity, the signal pulse duration and combinations thereof of the one or more electrical pulses. Additionally, the kit may include a CPU, a keyboard, a mouse, a touchpad, a touch screen, a Bluetooth wireless adaptor, an IR adaptor, a wi-fi adaptor, a RF adaptor, a blood pressure sensor, a heart rate sensor, an electrical activity sensor, a contraction sensor, a timer, speakers, a beeper, an input port, an output port, an IR sensor, a RF sensor, a biofeedback sensor, a LAN adaptor, wireless network adaptor and combinations thereof When the kit is in the form of modules the electrical energy generator may include modules that generates the signal, modules that control the signal, modules that connect the electrical energy generator to a CPU, a keyboard, a mouse, a touchpad, a touch screen, a Bluetooth wireless adaptor, an IR adaptor, a wi-fi adaptor, a RF adaptor, a blood pressure sensor, a heart rate sensor, an electrical activity sensor, a contraction sensor, a timer, speakers, a beeper, an input port, an output port, an IR sensor, a RF sensor, a biofeedback sensor, a LAN adaptor, wireless network adaptor and combinations thereof. Alternatively, each module may contain more that one function, e.g., an input port, output port, IR sensor, RF sensor module; a LAN adaptor, wireless network adaptor module and so forth.

In addition, the present invention provides a neuromodulation device for the reduction of preterm labor contractions having one or more percutaneous electrodes adapted for electrical communication with one or more dura layers surrounding one or more sacral nerve roots and an electrical energy generator to generate one or more electrical pulses in electrical communication with the one or more implantable electrodes.

The present invention includes a method of neuron-stimulation to reduce preterm labor by connecting one or more electrodes, under the control of a neuron-stimulation apparatus. The neuron-stimulation apparatus includes an electrical energy generator to stimulate one or more sacral nerves. The one or more electrodes are stimulated through the conduction of the one or more electrical pulses to the one or more electrodes. The method of neuron-stimulation to reduce preterm labor may further include controlling the pulse waveform, the signal pulse width, the signal pulse frequency, the signal pulse phase, the signal pulse polarity, the signal pulse amplitude, the signal pulse intensity, the signal pulse duration and combinations thereof of the one or more electrical pulses. Furthermore, the neuron-stimulation apparatus may include a CPU, a keyboard, a mouse, a touchpad, a touch screen, a Bluetooth wireless adaptor, an IR adaptor, a wi-fi adaptor, a RF adaptor, a blood pressure sensor, a heart rate sensor, an electrical activity sensor, a contraction sensor, a timer, speakers, a beeper, an input port, an output port, an IR sensor, a RF sensor, a biofeedback sensor, a LAN adaptor, wireless network adaptor and combinations thereof. Furthermore, the present invention may have a feedback system for measuring changes in the conductivity of the one or more electrodes during a discrete time period.

In addition, the neuron-stimulation apparatus may include one or more modules operatively coupled together, each one of the modules including one or more integrated circuit electrically connected to the electrodes for independently providing electrical current to each of the electrodes in a predetermined control sequence and a CPU or a PC board. The power may be supplied by an internal source or external source in the form of a battery, a generator or outlet plug.

Generally, a percutaneous electrode is a thin wire type electrode having a circular cross-section of about 0.05 inches; however, the skilled artisan will recognize that other size electrodes may be used. Typically, one or more equally-spaced ring electrodes are placed above the dura layer of a patient using a Touhy-like needle; however the number, position and spacing may depend on the specific requirements of the subject. It is not uncommon to insert 2, 3, 4, 5, 6, 7, 8, 9, 10 or more total electrodes into area. The Touhy-like needle is inserted into the spinal canal area between adjacent vertebrae until the tip is advanced into the epidural space of the spinal canal area. The wire lead is inserted through the open area or lumen of the Touhy-like needle and into the epidural space to a selected location adjacent to the spinal cord. In addition, the distal tip of the Touhy-like needle may be curved to facilitate introduction of the electrode at an angle to the axis of the lumen. In some instances, the Touhy-like needle is a needle assembly or a stylet assembly and may be contain a removable insert to fill the lumen cavity, including the opening of the needle, to prevent the collection of tissue in the lumen cavity during insertion and to provide rigidity to the needle body for use during insertion. Generally, the Touhy-like needle used for insertion of the electrode may have a circular cross section between 10 and 20 gauge; however the skilled artisan will recognize that other cross-sectional profiles and gauges may be used. The Touhy-like needle is passed through the skin, between desired vertebrae (e.g., S2, S3 and/or S4 ) and the percutaneous electrode is placed adjacent to the S2, S3 and/or S4 sacral nerve roots.

Laminotomy electrodes generally have a flat paddle configuration and typically possess a plurality of electrodes (e.g., 2, 3, 4, 5, 6, 7, 8, 9, 10 or more) arranged on the paddle. Although, the example presented of the Laminotomy electrodes is of a paddle configuration, the electrode may have any convenient shape and profile. The arrangement of the electrodes on the paddle may be in rows and columns, staggered, spaced, circular, or any other arrangement that will position the electrodes in the needed areas.

The specific configuration (e.g., size, shape, thickness, number of electrodes, spacing, etc.) of the laminotomy electrode may vary depending on the specific need. For example, the surface of the electrode may be paddle shaped with the paddle portion being about 0.4 inches wide and about 0.06 inches thick; however, the width may range from about 0.1 inches to about 1 inch and the thickness may range from about 0.01 to about 0.5 inches. Alternatively, the electrode may be a flat linear or curved electrode being about 0.3 inches wide and about 0.08 inches thick; however, the width may range from about 0.1 inches to about 1.5 inch and the thickness may range from about 0.01 to about 0.8 inches. Generally, the electrodes are exposed to one side of the paddle to dissipate the application of electrical energy.

In general, the paddle electrode or flat laminotomy electrodes is implanted into the desired vertebrae (e.g., S2, S3 and/or S4 ). For example, the center of the laminotomy electrode is positioned at about the midline to allow the electrodes of the paddle to contact the sacral nerve located about the S2, S3 and/or S4 vertebrae. In using the laminotomy electrodes the relative position of the laminotomy electrodes are maintained and in operation the various electrodes of the paddle may be used to create specific areas of stimulation. The laminotomy electrodes must be implanted using a surgical procedure that involves the removal of tissue to allow access to the dura and proper positioning of the electrodes. The surgical procedure allows the laminotomy electrodes to be positioned and decreases the migration of the electrode in addition it is possible to fix the position of the electrode using sutures.

In some instances, the electrodes may be connected to a simple stimulation system; however, the present invention also includes a multi-programmable neuromodulation system. Typically, the system includes a connection for each of the electrodes that allows the designation of the connected stimulation lead as an anode (+), a cathode (−), or in an OFF-state. This may be done in the form of interchangeable connections or through programs that electronically control the electrode to designate them individually as an anode (+), a cathode (−), or in an OFF-state. Generally, the electric current “flows” from an anode to a cathode. The variations of combinations of electrode states allow the concentration of electrical energy at a particular point or over a region. In some instances, the spinal nerve tissue may be more deeply located and require a more focused application of electrical energy to the nervous tissue to reach the deeply situated target nerve tissue and avoid undesirable stimulation of unafflicted regions.

The system may be of any convenient design, form small and portable having an internal battery that is replaceable or rechargeable to an institutional design having an external power supply and a CPU or under computer control to provide various activities and programs. In addition, the controls may be as simple as a knob or button to select electrodes, configurations (e.g., current, voltage, pulse width, cycle, frequency, amplitude and so forth), currents, voltages and times or as complicated as entry of the parameters using an input pad or computer. The parameters may be set using a pre-stored profile or storable profile either internally or externally to the system. In addition, the system may include connections for input and output devices including a keyboard, a mouse, a touchpad, a touch screen, a Bluetooth wireless adaptor, an IR adaptor, a wi-fi adaptor, a RF adaptor, and connections for numerous electrodes and sensors, e.g., blood pressure sensors, heart rate sensors, electrical activity sensors, contraction sensors, timers, speakers, beepers, input and output ports, IR sensors, RF sensors, biofeedback sensor and combinations thereof. It is also possible to connect the system of the present invention to a network or wireless system (e.g., LAN, wireless network, local “hotspot” networks, cellular networks, telephone networks, cable networks, satellite networks and combinations thereof) to allow constant monitoring of conditions and transfer of protocols to give the physician real-time information. The connection may be maintained constantly or intermittently depending on the particular application. Thus, the present invention provides the physician with information that can be used to make decisions regarding treatment.

In operation, the present invention may provide electrical energy to the sacral nerves associated with the spinal cord. The electrical energy may be in the form of a continuous signal, an intermittent signal or a pulsed signal in terms of signal, signal strength, signal frequency, signal phase, signal polarity and signal amplitude. The present invention may include a pulse generator (e.g., totally implanted or an RF-coupled nature) to deliver an electrical signal having a defining a signal waveform (e.g., signal pulse width, frequency, phase, polarity and amplitude) through one or more multi-electrode leads. Alternatively, the present invention includes an electrical pulse generator to generate an electrical pulse having a defining a pulse waveform, e.g., signal pulse width, frequency, phase, polarity and amplitude.

In addition, the present invention may include multi-electrode (e.g., 2, 3, 4, 5, 6 or more implants each having 2, 3, 4, 5, 6 or more electrodes) and a system to control the possible number of electrode combinations (e.g., combination of cathodes, anodes and off electrodes) and the waveform variations (e.g., signal pulse width, frequency, phase, polarity and amplitude) to optimize the therapeutic regimen.

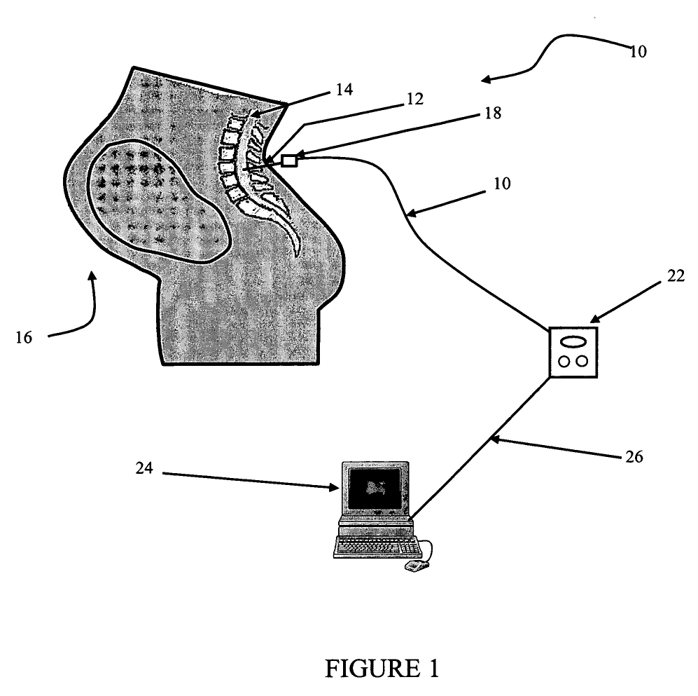

With reference to FIG. 1, a schematic view of the system connected to a patient. The system 10 includes one or more electrodes 12 inserted into the spine 14 at the sacral vertebrae of the patient 16. The electrodes 12 may be secured to the patient 16 and have connections 18 for connecting the electrodes 12 to the leads 20 to communicate with the electrical energy generator 22. In some embodiments, the electrical energy generator 22 may be part of a larger system (not shown) having inputs, outputs and sensors or individual modules (not shown). In addition, the present invention may be connected to a computer 24 or other control system (not shown) using a wireless connection (not shown) or a wired connection 26.

The components of the present invention may be constructed from any suitable similar singular or composite material, e.g., copper, silver, gold, a metal, an alloy, a steel, a composite, a polymer, a blend of polymers, a carbon fiber, a plastic, a thermoplastic, carbon nanotubes, a synthetic material or other material known to the skilled artisan, depending on the particular need or application. In addition, combinations and mixtures of material may be used, e.g., a polymer, a metal, a plastic, a fiber, a composite; a metal-coated polymer, metal, plastic, fiber, ceramic and/or composite; a carbon nanotube-coated polymer, metal, plastic, fiber and/or composite; a polymer-coated polymer, metal, plastic, fiber and/or composite; a magnetic material combined with a polymer, metal, plastic, fiber and/or composite; an electrical conductive material combined with a polymer, metal, plastic, fiber and/or composite; and so-forth. The materials used are not limited to the above noted and may also include other suitable solid materials that have the above-noted properties but are most often biocompatible. In some embodiments, the materials may even be biodegradable or bactericidal themselves or be coated or surrounded with a biodegradable or bactericidal agent. Additionally, the present invention may include a polymeric coating or layer on part or all of the surfaces that includes one or more bioactive substances, such as antibiotics, chemotherapeutic substances, angiogenic growth factors, substances for accelerating the healing of the wound, hormones, antithrombogenic agents, steroids, anti inflammatory agents, preterm labor reducing chemical agent known to the skilled artisan and the like. Often these substances will be provided for extended release.

In addition, the electrode of the present invention may take many different forms, e.g., a looped wire, a molded loop, a hook, a bent material, a fused material, a welded material, an epoxy material, a coated material or a doped material.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations can be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. An implantable neurostimulation apparatus to reduce preterm labor comprising:
one or more implantable electrodes adapted for electrical communication with one or more sacral nerve roots; and
an electrical energy generator to generate one or more electrical signals in electrical communication with the one or more implantable electrodes.

2. The apparatus of claim 1, wherein the one or more implantable electrodes comprises a wire, a rod, a filament, a ribbon, a cord, a formed wire, a flat strip, a tube or combination thereof.

3. The apparatus of claim 1, wherein the one or more implantable electrodes comprise one or more percutaneous electrodes, one or more laminotomy electrodes or a combination thereof.

4. The apparatus of claim 1, wherein each of the one or more implantable electrodes are controlled individually.

5. The apparatus of claim 1, wherein the one or more implantable electrodes are secured using stitches, epoxy, tape, glue, sutures or a combination thereof.

6. The apparatus of claim 1, further comprising one or more implantable electrodes adapted for electrical communication with one or more thoracic nerve roots, one or more lumbar nerve roots, one or more sacral nerve roots or combinations thereof.

7. The apparatus of claim 1, wherein the one or more sacral nerve roots comprise the S2 sacral nerve roots, the S3 sacral nerve roots, the S4 sacral nerve roots and combinations thereof.

8. The apparatus of claim 1, wherein the electrical energy generator controls the waveform, the signal width, the signal frequency, the signal phase, the signal polarity, the signal amplitude, the signal intensity, the signal duration and combinations thereof of the one or more electrical pulses.

9. The apparatus of claim 1, wherein the electrical energy generator further comprises a CPU, a keyboard, a mouse, a touchpad, a touch screen, a Bluetooth wireless adaptor, an IR adaptor, a wi-fi adaptor, a RF adaptor, a blood pressure sensor, a heart rate sensor, an electrical activity sensor, a contraction sensor, a timer, speakers, a beeper, an input port, an output port, an IR sensor, a RF sensor, a biofeedback sensor, a LAN adaptor, wireless network adaptor and combinations thereof.

10. An implantable neural stimulation kit for reduction of preterm labor comprising:
one or more percutaneous electrodes adapted for electrical communication with one or more nerve roots; and
an electrical energy generator to generate one or more electrical pulses in electrical communication with the one or more implantable electrodes.

11. The kit of claim 10, further comprising a Touhy-like needle.

12. A neuromodulation device for the reduction of preterm labor contractions comprising:
one or more percutaneous electrodes adapted for electrical communication with one or more dura layers surrounding one or more sacral nerve roots; and
an electrical energy generator to generate one or more electrical signals in electrical communication with the one or more implantable electrodes.

13. The device of claim 12, wherein each of the one or more percutaneous electrodes are controlled individually.

14. The device of claim 12, wherein the one or more percutaneous electrodes are secured using stitches, epoxy, tape, glue, sutures or a combination thereof.

15. The device of claim 12, wherein the one or more sacral nerve roots comprise the S2 sacral nerve roots, the S3 sacral nerve roots, the S4 sacral nerve roots and combinations thereof.

16. The device of claim 12, wherein the electrical energy generator controls the waveform, the signal width, the signal frequency, the signal phase, the signal polarity, the signal amplitude, the signal intensity, the signal duration and combinations thereof of the one or more electrical signals.

17. The device of claim 12, further comprising a CPU, a keyboard, a mouse, a touchpad, a touch screen, a Bluetooth wireless adaptor, an IR adaptor, a wi-fi adaptor, a RF adaptor, a blood pressure sensor, a heart rate sensor, an electrical activity sensor, a contraction sensor, a timer, speakers, a beeper, an input port, an output port, an IR sensor, a RF sensor, a biofeedback sensor, a LAN adaptor, wireless network adaptor and combinations thereof.

18. A method of neuron-stimulation to reduce preterm labor comprising the steps of:
connecting one or more electrodes under the control of a neuron-stimulation apparatus comprising an electrical energy generator to one or more sacral nerves; and
stimulating the one or more electrodes through the conduction of the one or more electrical pulses to the one or more electrodes.

19. The method of claim 18, wherein the one or more electrodes comprises a wire, a rod, a filament, a ribbon, a cord, a tube or combination thereof.

20. The method of claim 18, wherein the one or more electrodes comprises a percutaneous electrode, a laminotomy electrode or a combination thereof.

21. The method of claim 18, further comprising the step of controlling each of the one or more electrodes individually.

22. The method of claim 18, wherein the one or more sacral nerves comprise the S2 sacral nerve roots, the S3 sacral nerve roots, the S4 sacral nerve roots and combinations thereof.

23. The method of claim 18, further comprising the step of controlling the pulse waveform, the signal pulse width, the signal pulse frequency, the signal pulse phase, the signal pulse polarity, the signal pulse amplitude, the signal pulse intensity, the signal pulse duration and combinations thereof of the one or more electrical pulses.

24. The method of claim 18, wherein the neuron-stimulation apparatus further comprises a CPU, a storage device, a keyboard, a mouse, a touchpad, a touch screen, a Bluetooth wireless adaptor, an IR adaptor, a wi-fi adaptor, a RF adaptor, a blood pressure sensor, a heart rate sensor, an electrical activity sensor, a contraction sensor, a timer, speakers, a beeper, an input port, an output port, an IR sensor, a RF sensor, a biofeedback sensor, a LAN adaptor, wireless network adaptor and combinations thereof.
